(19) United States
(12) Patent Application Publication
(10) Pub. No.: US 2024/0391866 A1
(43) Pub. Date: Nov. 28, 2024

(54) PROCESS FOR THE PREPARATION OF THE SALBUTAMOL INTERMEDIATE

(71) Applicant: YEDA RESEARCH AND DEVELOPMENT CO. LTD., Rehovot (IL)

(72) Inventors: David MILSTEIN, Rehovot (IL); Kumar Das UTTAM, Rehovot (IL); Kar SAYAN, Rehovot (IL)

(73) Assignee: YEDA RESEARCH AND DEVELOPMENT CO. LTD., Rehovot (IL)

(21) Appl. No.: 18/692,847

(22) PCT Filed: Oct. 12, 2022
(86) PCT No.: PCT/IL2022/051083

§ 371 Date: Mar. 18, 2024

Related U.S. Application Data
(60) Provisional application No. 63/255,539, filed on Oct. 14, 2021.

(51) Int. Cl. C07C 217/70; B01J 31/18

(52) U.S. Cl. CPC C07C 217/70; B01J 31/1815; B01J 31/189; B01J 2231/643; B01J 2531/72

(57) ABSTRACT

This invention is directed to a catalytic hydrogenation process for the preparation of 1-(4-(benzyloxy)-3-(hydroxymethyl)phenyl)-2-(tert-butylamino)ethanol, which is an intermediate for the preparation of Salbutamol.

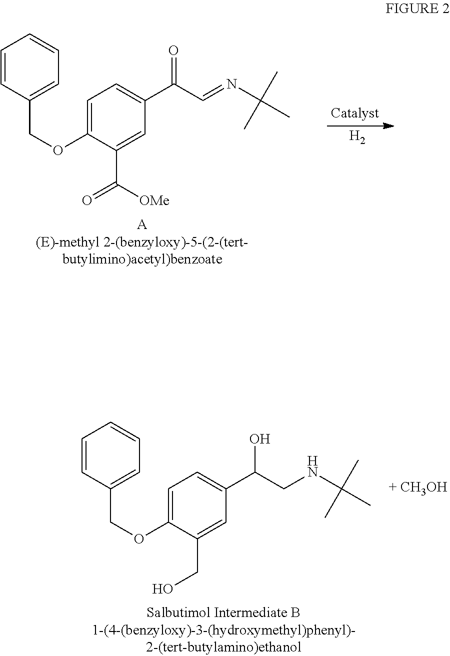

### FIELD OF THE INVENTION

This invention is directed to a catalytic hydrogenation process for the preparation of 1-(4-(benzyloxy)-3-(hydroxymethyl)phenyl)-2-(tert-butylamino)ethanol, an intermediate for the preparation of Salbutamol.

### BACKGROUND OF THE INVENTION

Salbutamol (4-(2-(tert-butylamino)-1-hydroxyethyl)-2-(hydroxymethyl)phenol) is a racemic mixture of the R- and S-isomers having the following structure:

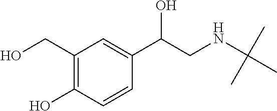 [SMILES: CC(C)(C)NCC(O)c1ccc(O)c(CO)c1]

Salbutamol is a short-acting, selective beta2-adrenergic receptor agonist and belongs to a class of drugs known as bronchodilators. It works in the airways by opening breathing passages and relaxing muscles. Salbutamol is used for (i) the symptomatic relief and prevention of bronchospasm due to bronchial asthma, chronic bronchitis, reversible obstructive airway disease, and other chronic bronchopulmonary disorders in which bronchospasm is a complicating factor, and/or (ii) the acute prophylaxis against exercise-induced bronchospasm and other stimuli known to induce bronchospasm. Salbutamol is used in the treatment of asthma and COPD.

Salbutamol is formulated as an inhaler and sometimes given as tablets, capsules or syrup for people who cannot use an inhaler very well.

One of the processes for the preparation of Salbutamol includes the reduction of Salbutamol intermediate [1-(4-(benzyloxy)-3-(hydroxymethyl)phenyl)-2-(tertbutylamino) ethan-1-ol]. Attempts to prepare this intermediate resulted with side products and impurities. For example, S. Ya. Skachilova, et al., Methods for the Preparation of Salbutamol (Review), Methods of Synthesis and Technology of the Production of Drugs, P. 733-739, 1992 includes a reaction of methyl (E)-2-(benzyloxy)-5-(2-(tert-butylimino)acetyl)benzoate as a starting material reacting with NaAl(OCH2CH2OCH3)H2 (VITRIDE) reagent (FIG. 1). This step requires reduction of 3 functionalities: ester, ketone and imine and uses a large amount of the well-known costly aluminum hydride-based Vitride reagent, generating a large amount of waste.

The inventors developed a superior process, in which the Salbutamol intermediate [1-(4-(benzyloxy)-3-(hydroxymethyl)phenyl)-2-(tertbutylamino) ethan-1-ol] is formed by catalytic hydrogenation of the same starting compound using catalysts, avoiding the use of the Vitride reagent (FIG. 2).

### SUMMARY OF THE INVENTION

This invention provides a process for the preparation of 1-(4-(benzyloxy)-3-(hydroxymethyl)phenyl)-2-(tert-butylamino)ethanol (Salbutamol Intermediate):

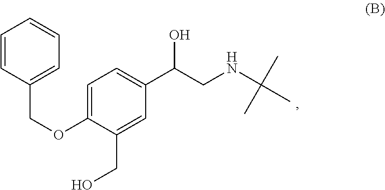 [SMILES: CC(C)(C)NCC(O)c1ccc(OCc2ccccc2)c(CO)c1]

wherein the process comprises reacting (E)-methyl 2-(benzyloxy)-5-(2-(tert butylimino)acetyl)benzoate:

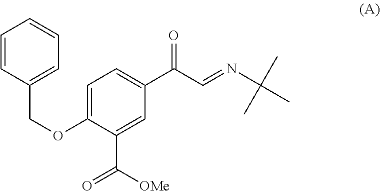 [SMILES: COC(=O)c1cc(C(=O)/C=N/C(C)(C)C)ccc1OCc1ccccc1]

with a catalyst represented by the structure of formula Ia or Ia′ under hydrogen pressure:

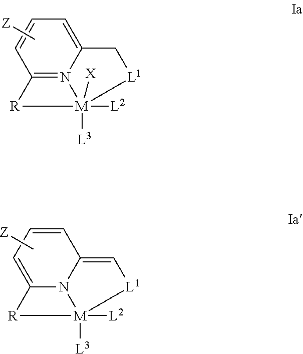

wherein,
M is a transition metal Ru(II) or Mn(I);
R is CH₂L⁴ wherein L⁴ is coordinated with the metal; or R is a substituted or unsubstituted pyridyl group, wherein the nitrogen of the pyridyl group is coordinated with the metal;
L¹ is (PR^{a}R^{b}), (NR^{a}R^{b}), imine; oxazoline, sulfide (SR^{a}), heteroaryl containing at least one heteroatom selected from nitrogen and sulfur; or a N-heterocyclic carbene represented by the structures:

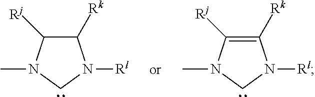

if M is Mn(I), L² and L³ are each independently a mono-dentate two-electron donor selected from the group consisting of CO, PR^{a}R^{b}R^{c}, P(OR^{a})(OR^{b})(OR^{c}), NO⁺, NR^{a}R^{b}R^{c}, AsR^{a}R^{b}R^{c}, SR^{a}R^{b}, nitrile (RCN), isonitrile (RNC), PF₃, CS, heteroaryl, tetrahydrothiophene, alkene or an alkyne; if M is Ru(II), L² is a mono-dentate two-electron donor selected from the group consisting of CO, PR^{a}R^{b}R^{c}, P(OR^{a})(OR^{b})(OR^{c}), NO⁺, NR^{a}R^{b}R^{c}, ASR^{a}R^{b}R^{c}, SR^{a}R^{b}, nitrile (RCN), isonitrile (RNC), PF₃, CS, heteroaryl, tetrahydrothiophene, alkene, alkyne; and L³ is H, halide, OCOR^{X}, OCH₂Q, OCOCF₃, OSO₂R^{X}, OSO₂CF₃, CN, OR^{X}, N(R^{X})₂ or R^{X}S; wherein Q is hydrogen, alkyl, cycloalkyl, aryl, heterocyclyl, heteroaryl, alkylcycloalkyl, alkylaryl, alkylheterocyclyl or alkylheteroaryl;
L⁴ is (PR^{a}R^{b}), (NR^{a}R^{b}), imine; oxazoline, sulfide (SR^{a}), heteroaryl containing at least one heteroatom selected from nitrogen and sulfur; (AsR^{a}R^{b}), or a N-heterocyclic carbene represented by the structures:

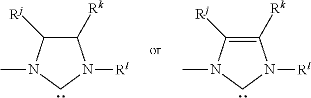

R^{j}, R^{k} and R^{l} are substituents of a N-heterocyclic carbene wherein each independently H, alkyl, cycloalkyl, aryl, heterocyclyl, heteroaryl, alkylcycloalkyl, alkylaryl, alkylheterocyclyl or alkylheteroaryl;
X is H, halide, OCOR^{X}, OCH₂Q, OCOCF₃, OSO₂R^{X}, OSO₂CF₃, CN, OR^{X}, N(R^{X})₂ or R^{X}S; wherein Q is hydrogen, alkyl, cycloalkyl, aryl, heterocyclyl, heteroaryl, alkylcycloalkyl, alkylaryl, alkylheterocyclyl or alkylheteroaryl;
R^{x} is hydrogen, alkyl, cycloalkyl, aryl, heterocyclyl, heteroaryl, alkylcycloalkyl, alkylaryl, alkylheterocyclyl or alkylheteroaryl;
Z represents zero, one, two or three substituents wherein each such substituent is independently selected from the group consisting of alkyl, cycloalkyl, aryl, heterocyclyl, heteroaryl, alkylcycloalkyl, alkylaryl, alkylheterocyclyl, alkylheteroaryl, halide, nitro, amide, ester, cyano, alkoxy, alkylamino, arylamino, an inorganic support and a polymeric moiety; or Z forms a fused aromatic or heterocyclic ring with the nitrogen based ring; and
R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl.

In other embodiments, the reaction is conducted with the exclusion of oxygen. In other embodiments, the reaction is conducted with the exclusion of air. In other embodiments, the reaction is conducted under hydrogen pressure is between 10-70 bars. In other embodiments, the reaction is conducted at a temperature of between 120-150° C. In other embodiments, the reaction is conducted in the presence of a strong base. In other embodiments, the molar ratio between (E)-methyl 2-(benzyloxy)-5-(2-(tert-butylimino)acetyl)benzoate (A) and the catalyst is between 100:1-20:1. In other embodiments, wherein the molar ratio between the catalyst and the base is 1:1.

In one embodiment, provided herein a process for the preparation of Salbutamol wherein the process comprises catalytic hydrogenation of (E)-methyl 2-(benzyloxy)-5-(2-(tert-butylimino)acetyl)benzoate (A), to obtain the Salbutamol intermediate (B) as described herein; and the Salbutamol intermediate (B) is further reduced to yield Salbutamol.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a synthetic scheme for the preparation of Salbutamol wherein the Salbutamol intermediate [1-(4-(benzyloxy)-3-(hydroxymethyl)phenyl)-2-(tertbutylamino) ethan-1-ol] is prepared using the vitride reagent. (prior art)

FIG. 2 is a synthetic scheme for the preparation of the Salbutamol intermediate [1-(4-(benzyloxy)-3-(hydroxymethyl)phenyl)-2-(tertbutylamino) ethan-1-ol], using the catalyst described herein.

FIG. 3 presents a single crystal X-ray structure of Mn(CO)₂PNN^{ipr}Br (Example 2).

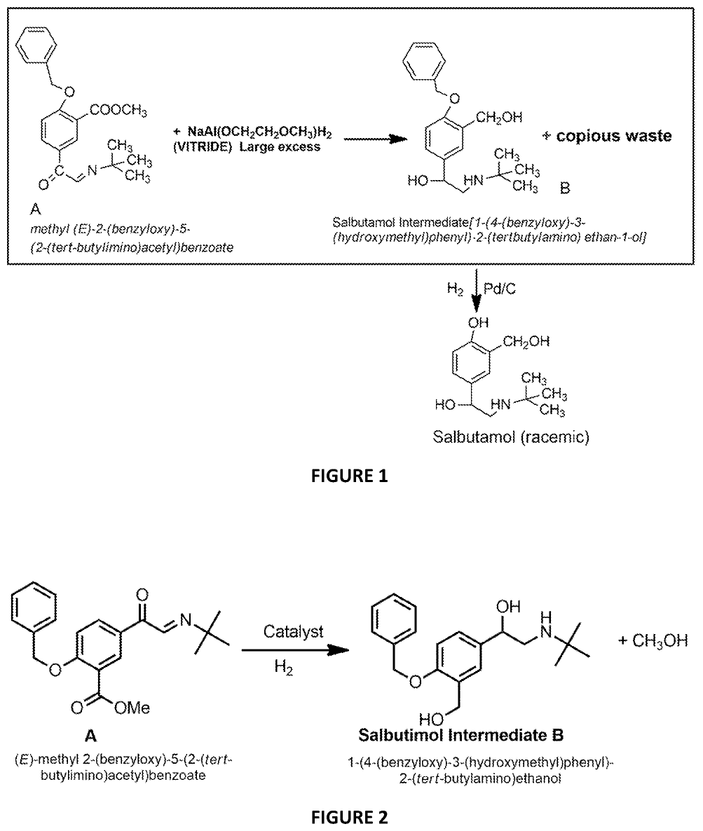

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, this invention provides a process for the preparation of 1-(4-(benzyloxy)-3-(hydroxymethyl)phenyl)-2-(tert-butylamino)ethanol (Salbutamol Intermediate) (B):

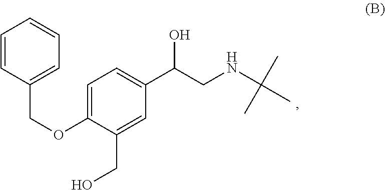 [SMILES: CC(C)(C)NCC(O)c1ccc(OCc2ccccc2)c(CO)c1]

wherein the process comprises reacting (E)-methyl 2-(benzyloxy)-5-(2-(tert-butylimino)acetyl)benzoate (A):

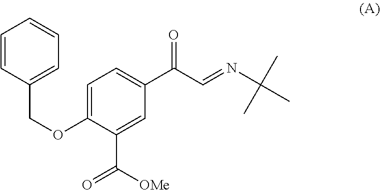 [SMILES: COC(=O)c1cc(C(=O)/C=N/C(C)(C)C)ccc1OCc1ccccc1]

with a catalyst represented by the structure of formula Ia or Ia′ under hydrogen pressure:

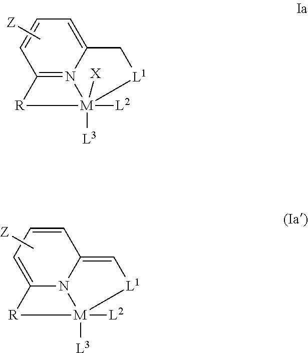

wherein,
M is a transition metal Ru(II) or Mn(I);
R is CH₂L⁴ wherein L⁴ is coordinated with the metal; or R is substituted or unsubstituted pyridyl group, wherein the nitrogen of the pyridyl group is coordinated with the metal;
L¹ is (PR^{a}R^{b}), (NR^{a}R^{b}), imine; oxazoline, sulfide (SR^{a}), heteroaryl containing at least one heteroatom selected from nitrogen and sulfur; or a N-heterocyclic carbene represented by the structures:

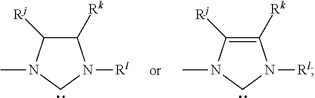

if M is Mn(I), L² and L³ are each independently a mono-dentate two-electron donor selected from the group consisting of CO, PR^{a}R^{b}R^{c}, P(OR^{a})(OR^{b})(OR^{c}), NO⁺, NR^{a}R^{b}R^{c}, AsR^{a}R^{b}R^{c}, SR^{a}R^{b}, nitrile (RCN), isonitrile (RNC), PF₃, CS, heteroaryl, tetrahydrothiophene, alkene, alkyne;
if M is Ru(II), L² is a mono-dentate two-electron donor selected from the group consisting of CO, PR^{a}R^{b}R^{c}, P(OR^{a})(OR^{b})(OR^{c}), NO⁺, NR^{a}R^{b}R^{c}, AsR^{a}R^{b}R^{c}, SR^{a}R^{b}, nitrile (RCN), isonitrile (RNC), PF₃, CS, heteroaryl, tetrahydrothiophene, alkene, alkyne; and L³ is H, halide, OCOR^{X}, OCH₂Q, OCOCF₃, OSO₂R^{X}, OSO₂CF₃, CN, OR^{X}, N(R^{X})₂ or R^{X}S; wherein Q is hydrogen, alkyl, cycloalkyl, aryl, heterocyclyl, heteroaryl, alkylcycloalkyl, alkylaryl, alkylheterocyclyl or alkylheteroaryl;
L⁴ is (PR^{a}R^{b}), (NR^{a}R^{b}), imine; oxazoline, sulfide (SR^{a}), heteroaryl containing at least one heteroatom selected from nitrogen and sulfur; (AsR^{a}R^{b}), or a N-heterocyclic carbene represented by the structures:

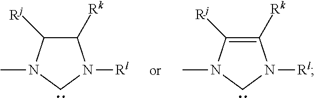

R^{j}, R^{k} and R^{l} are substituents of a N-heterocyclic carbene, wherein each independently H, alkyl, cycloalkyl, aryl, heterocyclyl, heteroaryl, alkylcycloalkyl, alkylaryl, alkylheterocyclyl or alkylheteroaryl;
X is H, halide, OCOR, OCH₂Q, OCOCF₃, OSO₂R^{X}, OSO₂CF₃, CN, OR^{X}, N(R^{X})₂ or R^{X}S; wherein Q is hydrogen, alkyl, cycloalkyl, aryl, heterocyclyl, heteroaryl, alkylcycloalkyl, alkylaryl, alkylheterocyclyl or alkylheteroaryl;
R^{X} is hydrogen, alkyl, cycloalkyl, aryl, heterocyclyl, heteroaryl, alkylcycloalkyl, alkylaryl, alkylheterocyclyl or alkylheteroaryl;
Z represents zero, one, two or three substituents wherein each such substituent is independently selected from the group consisting of alkyl, cycloalkyl, aryl, heterocyclyl, heteroaryl, alkylcycloalkyl, alkylaryl, alkylheterocyclyl, alkylheteroaryl, halide, nitro, amide, ester, cyano, alkoxy, alkylamino, arylamino, an inorganic support and a polymeric moiety; or Z forms a fused aromatic or heterocyclic ring with the nitrogen based ring; and
R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl.

In one embodiment, this invention provides a process for the preparation of 1-(4-(benzyloxy)-3-(hydroxymethyl)phenyl)-2-(tert-butylamino)ethanol (Salbutamol Intermediate) (B):

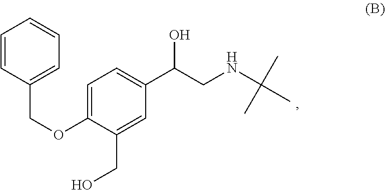 [SMILES: CC(C)(C)NCC(O)c1ccc(OCc2ccccc2)c(CO)c1]

wherein the process comprises reacting (E)-methyl 2-(benzyloxy)-5-(2-(tert-butylimino)acetyl)benzoate (A):

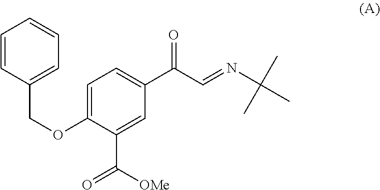 [SMILES: COC(=O)c1cc(C(=O)/C=N/C(C)(C)C)ccc1OCc1ccccc1]

with a catalyst represented by the structure of formula Ib or Ib′ under hydrogen pressure:

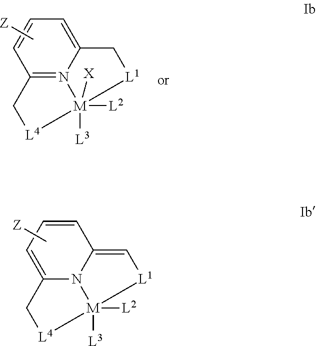

wherein M, L¹, L², L³, L⁴, X and Z are as described in the structure of formula Ia or Ia′.

In one embodiment, this invention provides a process for the preparation of 1-(4-(benzyloxy)-3-(hydroxymethyl)phenyl)-2-(tert-butylamino)ethanol (Salbutamol Intermediate) (B):

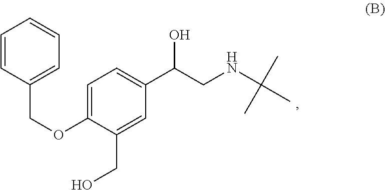 [SMILES: CC(C)(C)NCC(O)c1ccc(OCc2ccccc2)c(CO)c1]

wherein the process comprises reacting (E)-methyl 2-(benzyloxy)-5-(2-(tert-butylimino)acetyl)benzoate:

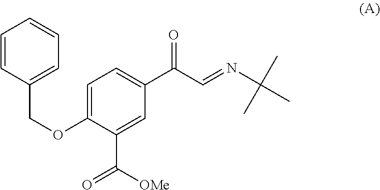 [SMILES: COC(=O)c1cc(C(=O)/C=N/C(C)(C)C)ccc1OCc1ccccc1]

with a catalyst represented by the structure of formula Ic or Ic′ under hydrogen pressure:

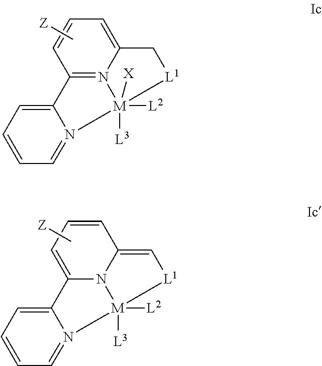

wherein M, L¹, L², L³, X and Z are as described in the structure of formula Ia or Ia′.

In other embodiments, the catalyst used in the processes of this invention is a Mn based catalyst represented by the structures of formula Id, Id′, Ie, or Ie′:

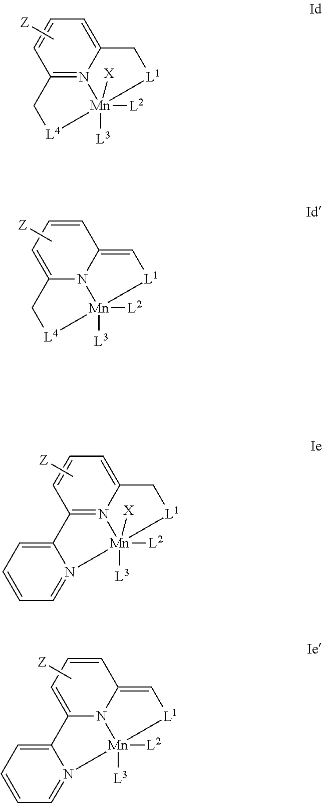

wherein L¹, L², L³, L⁴, X and Z are as described in the structure of formula Ia or Ia′.

In other embodiments, the catalyst used in the processes of this invention is a Mn based catalyst represented by the structures of formula If, If′, Ig or Ig′:

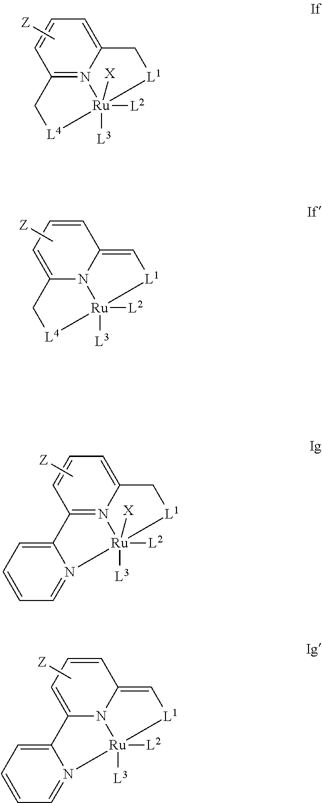

wherein L¹, L², L³, L⁴, X and Z are as described in the structure of formula Ia or Ia′.

In other embodiments, the catalyst used in the processes of this invention is represented by catalysts 1-5:

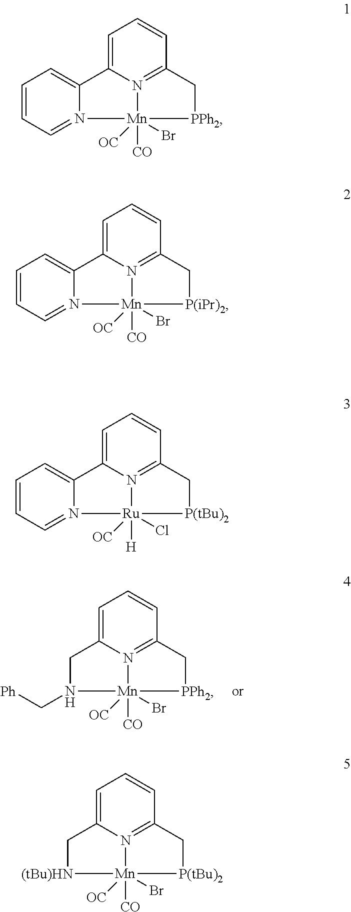

In some embodiment, M of formula Ia, Ia′, Ib, Ib′, Ic or Ic′ is a Ru(II). In some embodiment, M of formula Ia, Ia′, Ib, Ib′, Ic or Ic′ is a Mn(I) ion.

In some embodiment, R of formula Ia or Ia′ is CH₂L⁴ and L⁴ is coordinated with the metal or R is a substituted or unsubstituted pyridyl group, wherein the nitrogen of the pyridyl group is coordinated with the metal ion; each represents a separate embodiment according to this invention.

In some embodiments, L¹ of formula Ia, Ia′, Ib, Ib′ Ic, Ic′, Id, Id′, Ie, Ie′, If, If′, Ig or Ig′ is (PR^{a}R^{b}), (NR^{a}R^{b}), imine; oxazoline, sulfide (SR^{a}), heteroaryl containing at least one heteroatom selected from nitrogen and sulfuror a N-heterocyclic carbene represented by the structures:

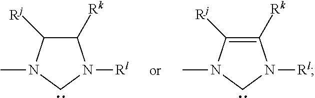

each represents a separate embodiment according to this invention;
wherein R^{j}, R^{k} and R^{l} are substituents of a N-heterocyclic carbene wherein each independently H, alkyl, cycloalkyl, aryl, heterocyclyl, heteroaryl, alkylcycloalkyl, alkylaryl, alkylheterocyclyl or alkylheteroaryl; and R^{a} and R^{b} are each independently, hydrogen alkyl, cycloalkyl, aryl, heterocyclyl, heteroaryl, alkylcycloalkyl, alkylaryl, alkylheterocyclyl or alkylheteroaryl. Each represents a separate embodiment according to this invention.

In some embodiments, L¹ of formula Ia, Ia′, Ib, Ib′ Ic, Ic′, Id, Id′, Ie, Ie′, If, If′, Ig or Ig′ is (PR^{a}R^{b}), wherein R^{a} and R^{b} are each independently, hydrogen alkyl, cycloalkyl, aryl, heterocyclyl, heteroaryl, alkylcycloalkyl, alkylaryl, alkylheterocyclyl or alkylheteroaryl. In some embodiments, L¹ of formula Ia, Ia′, Ib, Ib′ Ic, Ic′, Id, Id′, Ie, Ie′, If, If′, Ig or Ig′ is (NR^{a}R^{b}), wherein R^{a} and R^{b} are each independently, hydrogen alkyl, cycloalkyl, aryl, heterocyclyl, heteroaryl, alkylcycloalkyl, alkylaryl, alkylheterocyclyl or alkylheteroaryl. In some embodiments, L¹ of formula Ia, Ia′, Ib, Ib′ Ic, Ic′, Id, Id′, Ie, Ie′, If, If′, Ig or Ig′ is imine. In some embodiments, L¹ of formula Ia, Ia′, Ib, Ib′ Ic, Ic′, Id, Id′, Ie, Ie′, If, If′, Ig or Ig′ is oxazoline. In some embodiments, L¹ of formula Ia, Ia′, Ib, Ib′ Ic, Ic′, Id, Id′, Ie, Ie′, If, If′, Ig or Ig′ is sulfide (SR^{a}), wherein R^{a} is hydrogen alkyl, cycloalkyl, aryl, heterocyclyl, heteroaryl, alkylcycloalkyl, alkylaryl, alkylheterocyclyl or alkylheteroaryl. In some embodiments, L¹ of formula Ia, Ia′, Ib, Ib′ Ic, Ic′, Id, Id′, Ie, Ie′, If, If′, Ig or Ig′ is heteroaryl containing at least one heteroatom selected from nitrogen and sulfuror. In some embodiments, L¹ of formula Ia, Ia′, Ib, Ib′ Ic, Ic′, Id, Id′, Ie, Ie′, If, If′, Ig or Ig′ is a N-heterocyclic carbene represented by the structures:

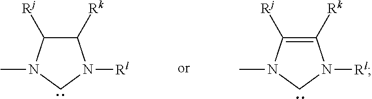

wherein R^{j}, R^{k} and R^{l}are substituents of a N-heterocyclic carbene wherein each independently H, alkyl, cycloalkyl, aryl, heterocyclyl, heteroaryl, alkylcycloalkyl, alkylaryl, alkylheterocyclyl or alkylheteroaryl. Each represents a separate embodiment according to this invention.

In some embodiments R^{j}, R^{k} and R^{l} are each independently a H. In some embodiments R^{j}, R^{k} and R^{l} are each independently an alkyl. In some embodiments R^{j}, R^{k} and R^{l} are each independently a cycloalkyl. In some embodiments R^{j}, R^{k} and R^{l} are each independently an aryl. In some embodiments R^{j}, R^{k} and R^{l} are each independently a heterocyclyl. In some embodiments R^{j}, R^{k} and R^{l} are each independently a heteroaryl. In some embodiments R^{j}, R^{k} and R^{l} are each independently an alkylcycloalkyl. In some embodiments R^{j}, R^{k} and R^{l} are each independently an alkylaryl. In some embodiments R^{j}, R^{k} and R^{l} are each independently an alkylheterocyclyl. In some embodiments R^{j}, R^{k} and R^{l} are each independently an alkylheteroaryl.

In some embodiments if M is Mn(I), L² and L³ of formula Ia, Ia′, Ib, Ib′ Ic, Ic′, Id, Id′, Ie, Ie′, If, If′, Ig or Ig′ is a mono-dentate two-electron donor selected from the group consisting of CO, PR^{a}R^{b}R^{c}, P(OR^{a})(OR^{b})(OR^{c}), NO⁺, NR^{a}R^{b}R^{c}, ASR^{a}R^{b}R^{c}, SR^{a}R^{b}, nitrile (RCN), isonitrile (RNC), PF₃, CS, heteroaryl, tetrahydrothiophene, alkene and an alkyne;
wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl, each represents a separate embodiment according to this invention.

In some embodiments if M is Mn(I), L² and L³ of formula Ia, Ia′, Ib, Ib′ Ic, Ic′, Id, Id′, Ie, Ie′, If, If′, Ig or Ig′ are each independently a CO. In some embodiments, L² and L³ of formula Ia, Ia′, Ib, Ib′ Ic, Ic′, Id, Id′, Ie, Ie′, If, If′, Ig or Ig′ are each independently a PR^{a}R^{b}R^{c}. In some embodiments, L² and L³ of formula Ia, Ia′, Ib, Ib′ Ic, Ic′, Id, Id′, Ie, Ie′, If, If′, Ig or Ig′ are each independently a P(OR^{a})(OR^{b})(OR^{c}). In some embodiments, L² and L³ of formula Ia, Ia′, Ib, Ib′ Ic, Ic′, Id, Id′, Ie, Ie′, If, If′, Ig or Ig′ are each independently a NO⁺. In some embodiments, L² and L³ of formula Ia, Ia′, Ib, Ib′ Ic, Ic′, Id, Id′, Ie, Ie′, If, If′, Ig or Ig′ are each independently a NR^{a}R^{b}R^{c}, wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl. In some embodiments, L² and L³ of formula Ia, Ia′, Ib, Ib′ Ic, Ic′, Id, Id′, Ie, Ie′, If, If′, Ig or Ig′ are each independently a AsR^{a}R^{b}R^{c}, wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl. In some embodiments, L² and L³ of formula Ia, Ia′, Ib, Ib′ Ic, Ic′, Id, Id′, Ie, Ie′, If, If′, Ig or Ig′ are each independently a SR^{a}R^{b}, wherein R^{a} and R^{b} are each independently hydrogen, alkyl, cycloalkyl, aryl, heterocyclyl, heteroaryl, alkylcycloalkyl, alkylaryl, alkylheterocyclyl or alkylheteroaryl. In some embodiments, L² and L³ of formula Ia, Ia′, Ib, Ib′ Ic, Ic′, Id, Id′, Ie, Ie′, If, If′, Ig or Ig′ are each independently a nitrile (RCN). In some embodiments, L² and L³ of formula Ia, Ia′, Ib, Ib′ Ic, Ic′, Id, Id′, Ie, Ie′, If, If′, Ig or Ig′ are each independently an isonitrile (RNC). In some embodiments, L² and L³ of formula Ia, Ia′, Ib, Ib′ Ic, Ic′, Id, Id′, Ie, Ie′, If, If′, Ig or Ig′ are each independently a PF₃. In some embodiments, L² and L³ of formula Ia, Ia′, Ib, Ib′ Ic, Ic′, Id, Id′, Ie, Ie′, If, If′, Ig or Ig′ are each independently a CS. In some embodiments, L² and L³ of formula Ia, Ia′, Ib, Ib′ Ic, Ic′, Id, Id′, Ie, Ie′, If, If′, Ig or Ig′ are each independently a heteroaryl. In some embodiments, L² and L³ of formula Ia, Ia′, Ib, Ib′ Ic, Ic′, Id, Id′, Ie, Ie′, If, If′, Ig or Ig′ are each independently a tetrahydrothiophene. In some embodiments, L² and L³ of formula Ia, Ia′, Ib, Ib′ Ic, Ic′, Id, Id′, Ie, Ie′, If, If′, Ig or Ig′ are each independently an alkene. In some embodiments, L² and L³ of formula Ia, Ia′, Ib, Ib′ Ic, Ic′, Id, Id′, Ie, Ie′, If, If′, Ig or Ig′ are each independently an alkyne.

In some embodiments if M is Ru(II), L² of formula Ia, Ia′, Ib, Ib′ Ic, Ic′, Id, Id′, Ie, Ie′, If, If′, Ig or Ig′ is a mono-dentate two-electron donor selected from the group consisting of CO, PR^{a}R^{b}R^{c}, P(OR^{a})(OR^{b})(OR^{c}), NO⁺, NR^{a}R^{b}R^{c}, ASR^{a}R^{b}R^{c}, SR^{a}R^{b}, nitrile (RCN), isonitrile (RNC), PF₃, CS, heteroaryl, tetrahydrothiophene, alkene and alkyne; and L³ is H, halide, OCOR^{X}, OCH₂Q, OCOCF₃, OSO₂R^{X}, OSO₂CF₃, CN, OR^{X}, N(R^{X})₂ or R^{X}S; wherein Q is hydrogen, alkyl, cycloalkyl, aryl, heterocyclyl, heteroaryl, alkylcycloalkyl, alkylaryl, alkylheterocyclyl or alkylheteroaryl; and R^{X} is hydrogen, alkyl, cycloalkyl, aryl, heterocyclyl, heteroaryl, alkylcycloalkyl, alkylaryl, alkylheterocyclyl or alkylheteroaryl; wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl, each represents a separate embodiment according to this invention.

In some embodiments if M is Ru(II), L² of formula Ia, Ia′, Ib, Ib′ Ic, Ic′, Id, Id′, Ie, Ie′, If, If′, Ig or Ig′ is a CO. In some embodiments L² of formula Ia, Ia′, Ib, Ib′ Ic, Ic′, Id, Id′, Ie, Ie′, If, If′, Ig or Ig′ is a PR^{a}R^{b}R^{c}, wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl. In some embodiments L² of formula Ia, Ia′, Ib, Ib′ Ic, Ic′, Id, Id′, Ie, Ie′, If, If′, Ig or Ig′ is a P(OR^{a})(OR^{b})(OR^{c}), wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl. In some embodiments L² of formula Ia, Ia′, Ib, Ib′ Ic, Ic′, Id, Id′, Ie, Ie′, If, If′, Ig or Ig′ is a NO⁺. In some embodiments L² of formula Ia, Ia′, Ib, Ib′ Ic, Ic′, Id, Id′, Ie, Ie′, If, If′, Ig or Ig′ is a NR^{a}R^{b}R^{c}, wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl. In some embodiments L² of formula Ia, Ia′, Ib, Ib′ Ic, Ic′, Id, Id′, Ie, Ie′, If, If′, Ig or Ig′ is a AsR^{a}R^{b}R^{c}, wherein R^{a}, R^{b} and R^{c} are each independently hydrogen, alkyl, cycloalkyl, aryl, heterocyclyl, heteroaryl, alkylcycloalkyl, alkylaryl, alkylheterocyclyl or alkylheteroaryl. In some embodiments L² of formula Ia, Ia′, Ib, Ib′ Ic, Ic′, Id, Id′, Ie, Ie′, If, If′, Ig or Ig′ is a SR^{a}R^{b}, wherein R^{a} and R^{b} are each independently hydrogen, alkyl, cycloalkyl, aryl, heterocyclyl, heteroaryl, alkylcycloalkyl, alkylaryl, alkylheterocyclyl or alkylheteroaryl. In some embodiments L² of formula Ia, Ia′, Ib, Ib′ Ic, Ic′, Id, Id′, Ie, Ie′, If, If′, Ig or Ig′ is a nitrile (RCN). In some embodiments L² of formula Ia, Ia′, Ib, Ib′ Ic, Ic′, Id, Id′, Ie, Ie′, If, If′, Ig or Ig′ is an isonitrile (RNC). In some embodiments L² of formula Ia, Ia′, Ib, Ib′ Ic, Ic′, Id, Id′, Ie, Ie′, If, If′, Ig or Ig′ is a PF₃. In some embodiments L² of formula Ia, Ia′, Ib, Ib′ Ic, Ic′, Id, Id′, Ie, Ie′, If, If′, Ig or Ig′ is a CS. In some embodiments L² of formula Ia, Ia′, Ib, Ib′ Ic, Ic′, Id, Id′, Ie, Ie′, If, If′, Ig or Ig′ is a heteroaryl. In some embodiments L² of formula Ia, Ia′, Ib, Ib′ Ic, Ic′, Id, Id′, Ie, Ie′, If, If′, Ig or Ig′ is a tetrahydrothiophene. In some embodiments L² of formula Ia, Ia′, Ib, Ib′ Ic, Ic′, Id, Id′, Ie, Ie′, If, If′, Ig or Ig′ is an alkene. In some embodiments L² of formula Ia, Ia′, Ib, Ib′ Ic, Ic′, Id, Id′, Ie, Ie′, If, If′, Ig or Ig′ is an alkyne.

In some embodiments if M is Ru(II), L³ of formula Ia, Ia′, Ib, Ib′ Ic, Ic′, Id, Id′, Ie, Ie′, If, If′, Ig or Ig′ is a H. In some embodiments L³ of formula Ia, Ia′, Ib, Ib′ Ic, Ic′, Id, Id′, Ie, Ie′, If, If′, Ig or Ig′ is a halide. In some embodiments L³ of formula Ia, Ia′, Ib, Ib′ Ic, Ic′, Id, Id′, Ie, Ie′, If, If′, Ig or Ig′ is a OCOR^{X}, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiments L³ of formula Ia, Ia′, Ib, Ib′ Ic, Ic′, Id, Id′, Ie, Ie′, If, If′, Ig or Ig′ is a OCH₂Q, wherein Q is hydrogen, alkyl, cycloalkyl, aryl, heterocyclyl, heteroaryl, alkylcycloalkyl, alkylaryl, alkylheterocyclyl or alkylheteroaryl. In some embodiments L³ of formula Ia, Ia′, Ib, Ib′ Ic, Ic′, Id, Id′, Ie, Ie′, If, If′, Ig or Ig′ is a OCOCF₃. In some embodiments L³ of formula Ia, Ia′, Ib, Ib′ Ic, Ic′, Id, Id′, Ie, Ie′, If, If′, Ig or Ig′ is a OSO₂R^{X}, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiments L³ of formula Ia, Ia′, Ib, Ib′ Ic, Ic′, Id, Id′, Ie, Ie′, If, If′, Ig or Ig′ is a OSO₂CF₃. In some embodiments L³ of formula Ia, Ia′, Ib, Ib′ Ic, Ic′, Id, Id′, Ie, Ie′, If, If′, Ig or Ig′ is a CN. In some embodiments L³ of formula Ia, Ia′, Ib, Ib′ Ic, Ic′, Id, Id′, Ie, Ie′, If, If′, Ig or Ig′ is a OR^{X}, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiments L³ of formula Ia, Ia′, Ib, Ib′ Ic, Ic′, Id, Id′, Ie, Ie′, If, If′, Ig or Ig′ is a N(R^{X})₂, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiments L³ of formula Ia, Ia′, Ib, Ib′ Ic, Ic′, Id, Id′, Ie, Ie′, If, If′, Ig or Ig′ is a R^{X}S, wherein R^{X} is hydrogen, alkyl, cycloalkyl, aryl, heterocyclyl, heteroaryl, alkylcycloalkyl, alkylaryl, alkylheterocyclyl or alkylheteroaryl.

In some embodiment, L⁴ of formula Ia, Ia′, Ib, Ib′, Id, Id′, If or If′ is (PR^{a}R^{b}), (NR^{a}R^{b}), imine; oxazoline, sulfide (SR^{a}), heteroaryl containing at least one heteroatom selected from nitrogen and sulfur; (AsR^{a}R^{b}), or a N-heterocyclic carbene represented by the structures:

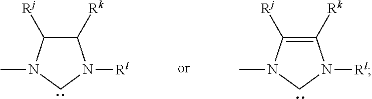

wherein R^{j}, R^{k} and R^{l} are substituents of a N-heterocyclic carbene wherein each independently H, alkyl, cycloalkyl, aryl, heterocyclyl, heteroaryl, alkylcycloalkyl, alkylaryl, alkylheterocyclyl or alkylheteroaryl; and R^{a} and R^{b} are each independently hydrogen, alkyl, cycloalkyl, aryl, heterocyclyl, heteroaryl, alkylcycloalkyl, alkylaryl, alkylheterocyclyl or alkylheteroaryl, each represents a separate embodiment according to this invention.

In some embodiment, L⁴ of formula Ia, Ia′, Ib, Ib′, Id, Id′, If or If′ is (PR^{a}R^{b}), wherein R^{a} and R^{b} are each independently hydrogen, alkyl, cycloalkyl, aryl, heterocyclyl, heteroaryl, alkylcycloalkyl, alkylaryl, alkylheterocyclyl or alkylheteroaryl. In some embodiment, L⁴ of formula Ia, Ia′, Ib, Ib′, Id, Id′, If or If′ is (NR^{a}R^{b}), wherein R^{a} and R^{b} are each independently hydrogen, alkyl, cycloalkyl, aryl, heterocyclyl, heteroaryl, alkylcycloalkyl, alkylaryl, alkylheterocyclyl or alkylheteroaryl. In some embodiment, L⁴ of formula Ia, Ia′, Ib, Ib′, Id, Id′, If or If′ is an imine. In some embodiment, L⁴ of formula Ia, Ia′, Ib, Ib′, Id, Id′, If or If′ is an oxazoline. In some embodiment, L⁴ of formula Ia, Ia′, Ib, Ib′, Id, Id′, If or If′ is a sulfide (SR^{a}), wherein R^{a} is hydrogen, alkyl, cycloalkyl, aryl, heterocyclyl, heteroaryl, alkylcycloalkyl, alkylaryl, alkylheterocyclyl or alkylheteroaryl. In some embodiment, L⁴ of formula Ia, Ia′, Ib, Ib′, Id, Id′, If or If′ is heteroaryl containing at least one heteroatom selected from nitrogen and sulfur. In some embodiment, L⁴ of formula Ia, Ia′, Ib, Ib′, Id, Id′, If or If′ is (AsR^{a}R^{b}), wherein R^{a} and R^{b} are each independently hydrogen, alkyl, cycloalkyl, aryl, heterocyclyl, heteroaryl, alkylcycloalkyl, alkylaryl, alkylheterocyclyl or alkylheteroaryl. In some embodiment, L⁴ of formula Ia, Ia′, Ib, Ib′, Id, Id′, If or If′ is a N-heterocyclic carbene represented by the structures:

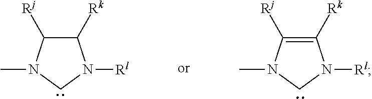

wherein R^{j}, R^{k} and R^{l} are substituents of a N-heterocyclic carbene wherein each independently H, alkyl, cycloalkyl, aryl, heterocyclyl, heteroaryl, alkylcycloalkyl, alkylaryl, alkylheterocyclyl or alkylheteroaryl.

In some embodiment, X of formula Ia, Ib, Ic, Id, Ie, If, or Ig is H, halide, OCOR^{X}, OCH₂Q, OCOCF₃, OSO₂R^{X}, OSO₂CF₃, CN, OR^{X}, N(R^{X})₂ or R^{X}S; wherein Q is hydrogen, alkyl, cycloalky, aryl, heterocyclyl, heteroaryl, alkylcycloalkyl, alkylaryl, alkylheterocyclyl or alkylheteroaryl; and R^{X} is hydrogen, alkyl, cycloalkyl, aryl, heterocyclyl, heteroaryl, alkylcycloalkyl, alkylaryl, alkylheterocyclyl or alkylheteroaryl each represents a separate embodiment according to this invention.

In some embodiment, X of formula Ia, Ib, Ic, Id, Ie, If, or Ig is H. In some embodiment, X of formula Ia, Ib, Ic, Id, Ie, If, or Ig is halide. In some embodiment, X of formula Ia, Ib, Ic, Id, Ie, If, or Ig is OCOR^{X}, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiment, X of formula Ia, Ib, Ic, Id, Ie, If, or Ig is OCH₂Q, wherein Q is hydrogen, alkyl, cycloalkyl, aryl, heterocyclyl, heteroaryl, alkylcycloalkyl, alkylaryl, alkylheterocyclyl or alkylheteroaryl. In some embodiment, X of formula Ia, Ib, Ic, Id, Ie, If, or Ig is OCOCF₃. In some embodiment, X of formula Ia, Ib, Ic, Id, Ie, If, or Ig is OSO₂R^{X}, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiment, X of formula Ia, Ib, Ic, Id, Ie, If, or Ig is OSO₂CF₃. In some embodiment, X of formula Ia, Ib, Ic, Id, Ie, If, or Ig is CN. In some embodiment, X of formula Ia, Ib, Ic, Id, Ie, If, or Ig is OR^{X}, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiment, X of formula Ia, Ib, Ic, Id, Ie, If, or Ig is N(R^{X})₂, wherein R^{X} is hydrogen, alkyl, cycloalkyl, aryl, heterocyclyl, heteroaryl, alkylcycloalkyl, alkylaryl, alkylheterocyclyl or alkylheteroaryl. In some embodiment, X of formula Ia, Ib, Ic, Id, Ie, If, or Ig is R^{X}S, wherein R^{X} is hydrogen, alkyl, cycloalkyl, aryl, heterocyclyl, heteroaryl, alkylcycloalkyl, alkylaryl, alkylheterocyclyl or alkylheteroaryl.

In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently selected from the group consisting of alkyl, cycloalkyl, aryl, heterocyclyl, heteroaryl, alkylcycloalkyl, alkylaryl, alkylheterocyclyl, alkylheteroaryl, halide, nitro, amide, ester, cyano, alkoxy, alkylamino, arylamino, an inorganic support and a polymeric moiety; or Z forms a fused aromatic or heterocyclic ring with the nitrogen based ring, each represents a separate embodiment according to this invention.

In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an alkyl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently a cycloalkyl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an aryl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently a heterocyclyl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently a heteroaryl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an alkylcycloalkyl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an alkylaryl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an alkylheterocyclyl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an alkylheteroaryl. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently a halide. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently a nitro. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an amide. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an ester. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently a cyano. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an alkoxy. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an alkylamino. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an arylamino. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently an inorganic support. In some embodiment, Z of formula Ia, Ia′, Ib, Ib′ Ic, Ic′, Id, Id′, Ie, Ie′, If, If′, Ig or Ig′ represents zero, one, two or three substituents wherein each such substituent is independently a polymeric moiety. In some embodiment, Z of formula Ia, Ia′, Ib, Ib′ Ic, Ic′, Id, Id′, Ie, Ie′, If, If′, Ig or Ig′ forms a fused aromatic or heterocyclic ring with the nitrogen based ring.

### Chemical Definitions

As used herein, the term alkyl, used alone or as part of another group, refers, in one embodiment, to a “C₁ to C₁₂ alkyl” and denotes linear and branched, saturated or unsaturated (e.g., alkenyl, alkynyl) groups, the latter only when the number of carbon atoms in the alkyl chain is greater than or equal to two, and can contain mixed structures. Non-limiting examples are alkyl groups containing from 1 to 6 carbon atoms (C₁ to C₆ alkyls), or alkyl groups containing from 1 to 4 carbon atoms (C₁ to C₄ alkyls). Examples of saturated alkyl groups include, but are not limited to, methyl, ethyl, n-propyl, isopropyl, n-butyl, iso-butyl, sec-butyl, tert-butyl, amyl, tert-amyl and hexyl. Examples of alkenyl groups include, but are not limited to, vinyl, allyl, butenyl and the like. Examples of alkynyl groups include, but are not limited to, ethynyl, propynyl and the like. Similarly, the term “C₁ to C₁₂ alkylene” denotes a bivalent radical of 1 to 12 carbons.

The alkyl group can be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups. Any substituents can be unsubstituted or further substituted with any one of these aforementioned substituents. By way of illustration, an “alkoxyalkyl” is an alkyl that is substituted with an alkoxy group.

The term “cycloalkyl” used herein alone or as part of another group, refers to a “C₃ to C₈ cycloalkyl” and denotes any unsaturated or unsaturated (e.g., cycloalkenyl, cycloalkynyl) monocyclic or polycyclic group. Nonlimiting examples of cycloalkyl groups are cyclopropyl, cyclobutyl, cyclopentyl, cyclohexyl or cycloheptyl. Examples or cycloalkenyl groups include cyclopentenyl, cyclohexenyl and the like. The cycloalkyl group can be unsubstituted or substituted with any one or more of the substituents defined above for alkyl. Similarly, the term “cycloalkylene” means a bivalent cycloalkyl, as defined above, where the cycloalkyl radical is bonded at two positions connecting together two separate additional groups.

The term “aryl” used herein alone or as part of another group denotes an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubtituted or substituted through available carbon atoms with one or more groups defined hereinabove for alkyl. An alkylaryl group denotes an alkyl group bonded to an aryl group (e.g., benzyl).

The term “heteroaryl” used herein alone or as part of another group denotes a heteroaromatic system containing at least one heteroatom ring atom selected from nitrogen, sulfur and oxygen. The heteroaryl contains 5 or more ring atoms. The heteroaryl group can be monocyclic, bicyclic, tricyclic and the like. Also included in this expression are the benzoheterocyclic rings. If nitrogen is a ring atom, the present invention also contemplates the N-oxides of the nitrogen containing heteroaryls. Nonlimiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like. The heteroaryl group can be unsubtituted or substituted through available atoms with one or more groups defined hereinabove for alkyl.

The term “heterocyclic ring” or “heterocyclyl” used herein alone or as part of another group denotes a five-membered to eight-membered rings that have 1 to 4 heteroatoms, such as oxygen, sulfur and/or nitrogen. These five-membered to eight-membered rings can be saturated, fully unsaturated or partially unsaturated. Non-limiting examples of heterocyclic rings include piperidinyl, piperidinyl, pyrrolidinyl pyrrolinyl, pyrazolinyl, pyrazolidinyl, piperidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, and the like. The heterocyclyl group can be unsubtituted or substituted through available atoms with one or more groups defined hereinabove for alkyl.

In one embodiment, the process of this invention make use of a Ru or Mn based metal complex as a catalyst. Thus, the Ru or Mn based metal complex is used in a catalytic amount in the processes of this invention. A catalytic amount refers to a significantly smaller amount of the catalyst than the molecular amount of substrates.

In some embodiments, the process of this invention is conducted under hydrogen pressure. In other embodiments, the hydrogen pressure is between 10-70 bars. In other embodiments, the hydrogen pressure is between 20-70 bars. In other embodiments, the hydrogen pressure is between 30-70 bars. In other embodiments, the hydrogen pressure is between 30-50 bars.

In some embodiments, the process of this invention comprises reacting (E)-methyl 2-(benzyloxy)-5-(2-(tert butylimino)acetyl)benzoate (A) under pressure of hydrogen with a catalyst Ia, Ib, Ic, Id, Ie or If described herein in the presence of a strong base. Non limiting examples of a strong base include lithium hydroxide, sodium hydroxide, potassium hydroxide, sodium ethoxide, potassium tert-butoxide, sodium methoxide. In one embodiment, the base is an organic base. In one embodiment, the base is an inorganic base. In other embodiments, the molar ratio between the catalyst and the base is 1:1.

In some embodiments, the process of this invention comprises reacting (E)-methyl 2-(benzyloxy)-5-(2-(tert butylimino)acetyl)benzoate (A) under pressure of hydrogen with a catalyst I′, Ia′, Ib′, Ic′, Id′, Ie′ or If′ described herein, without a base (no base is required).

In other embodiments, the molar ratio between (E)-methyl 2-(benzyloxy)-5-(2-(tert-butylimino)acetyl)benzoate (A) and the catalyst is between 100:1 to 10:1. In other embodiments, the molar ratio between (E)-methyl 2-(benzyloxy)-5-(2-(tert-butylimino)acetyl)benzoate (A) and the catalyst is between 1000:1, 500:1, 400:1, 300:1, 200:1, 100:1, 90:1, 80:1, 70:1, 60:1, 50:1, 40:1, 30:1, 20:1, 10:1 or any ranges thereof.

In some embodiments, the process of this invention is conducted with exclusion of oxygen. In some embodiments, the process of this invention is conducted with exclusion of air.

In some embodiments, the process of this invention is conducted at a temperature between 120° C. to 150° C. In other embodiments, the process of this invention is conducted at a temperature between 120° C. to 130° C. In other embodiments, the process of this invention is conducted at a temperature between 120° C. to 140° C.

In some embodiment this invention provides a process for the preparation of Salbutamol, wherein the process comprises reduction of Salbutamol intermediate 1-(4-(benzyloxy)-3-(hydroxymethyl)phenyl)-2-(tert-butylamino)ethanol (B):

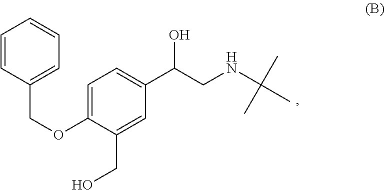 [SMILES: CC(C)(C)NCC(O)c1ccc(OCc2ccccc2)c(CO)c1]

wherein the Salbutamol intermediate is prepared according to the process described herein. In other embodiments, the reduction of the Salbutamol intermediate to obtain Salbutamol is done by any known process known in the art to remove a benzyl group to obtain an alcohol, for example by hydrogenation. In other embodiments by hydrogenation using H₂, Pd/C.

The following examples are presented in order to more fully illustrate the preferred embodiments of the invention. They should in no way be construed, however, as limiting the broad scope of the invention.

### EXAMPLES

### Example 1

General Procedure for the Synthesis of the Salbutamol Intermediate (B) [1-(4-(benzyloxy)-3-(hydroxymethyl) phenyl)-2-(tertbutylamino) ethan-1-ol] by Catalytic Hydrogenation

In a N₂ glove box, 0.02 mmol of the catalyst (as mentioned in Table 1) and 0.02 mmol of ^{t}BuOK were added in 4 mL of THF to a 20 mL vial. This mixture was stirred for 3 min, then 1 mmol of the starting compound methyl (E)-2-(benzyloxy)-5-(2-(tert-butylimino)acetyl)benzoate (A) was added to it and it was transferred to a steel autoclave fitted with a Teflon sleeve. The autoclave was taken out of the glove box and pressurized with hydrogen gas (pressure as specified in Table 1) and heated at the specified temperature with stirring (as specified in Table 1), after which the steel autoclave was cooled in an ice-bath for 30 min and the H₂ was vented off carefully. The cold solution was then filtered through Celite and the solution was analyzed by ¹H NMR spectroscopy.

The conversion of the starting compound (A) and the yields of partially hydrogenated compound (A1), (methyl 2-(benzyloxy)-5-(2-(tert-butylamino)-1-hydroxyethyl)benzoate, in which the ester group remained unreacted) and the desired Salbutamol Intermediate (B) were determined by ¹H NMR spectroscopy. In the reactions where the desired Salbutamol Intermediate (B) was quantitatively formed, it was obtained pure after removing the solvent and the biproduct methanol in vacuo.

Procedure with Mn Catalyst 1 (Table 1, Entry 4)

In a N₂ glove box, 0.02 mmol (11 mg) of Mn catalyst 1 and 0.02 mmol of ^{t}BuOK (2.2 mg) were added in 4 mL of THF to a 20 mL vial. This mixture was stirred for 3 min, then 1 mmol (353mg) of the starting compound methyl (E)-2-(benzyloxy)-5-(2-(tert-butylimino)acetyl)benzoate (A) was added to the solution and the solution was transferred to a steel autoclave fitted with a Teflon sleeve. The autoclave was taken out of the glove box and pressurized with 30 bar H₂ pressure and was heated at 130° C. with stirring for 36 hours. Afterwards, the steel autoclave was cooled in an ice-bath for 30 min and the H₂ was vented off slowly. The cold solution was then filtered through Celite, and the solution was analyzed by ¹H NMR spectroscopy. Pure Salbutamol intermediate B was obtained after removing the solvent THF and the byproduct methanol in vacuo.

¹H NMR (400 MHZ, CDCl₃) δ 7.50-7.31 (m, 6H), 7.27 (d, J=8.2 Hz, 1H), 6.93 (d, J=8.4 Hz, 1H), 5.12 (s, 2H), 4.75 (s, 2H), 4.57 (dd, J=8.7, 2.8 Hz, 1H), 2.85 (dd, J=11.7, 3.2 Hz, 1H), 2.67-2.55 (m, 1H), 1.11 (s, 9H).

¹³C NMR (101 MHZ, CDCl₃) δ 155.81, 136.83, 135.50, 129.60, 128.69, 128.08, 127.28, 126.30, 126.15, 111.48, 72.03, 70.15, 61.81, 50.32, 50.31, 29.18.

TABLE 1
Conditions of the process for the preparation of Salbutamol Intermediate (B).

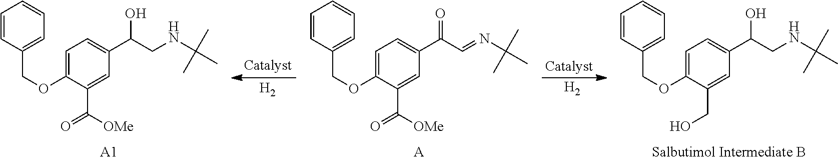

| | | Mol % (Cat)/ | H2 | Temp | Time | |
|---|---|---|---|---|---|---|
| Entry | Catalyst | mol % (^{t}BuOK) | (bar) | (° C.) | (H) | Product/yield |
| 1. | | 5/5 | 40 | 135 | 72 | B (99%) |
| | | | | | | 94% isolated |
| | | | | | | yield |
| 2. | | 5/5 | 40 | 135 | 72 | B (99%) |
| 3. | | 2/2 | 40 | 130 | 65 | B (99%) |
| 4. | | 2/2 | 30 | 130 | 36 | B (99%) |
| 5. | | 2/2 | 30 | 130 | 36 | A1 (75%) |
| 6. | 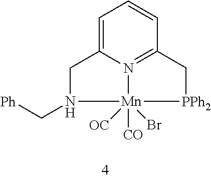 | 2/2 | 30 | 130 | 36 | 10% conversion |
| 7. | 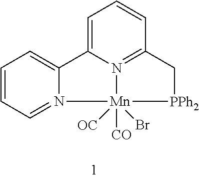 | 4/4 | 20 | 120 | 24 | A1(40%) |
| 8. | 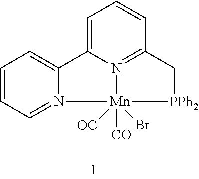 | 2/2 | 20 | 130 | 45 | A1 (55%) |
| 9. | 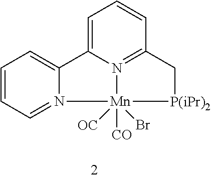 | 2/2 | 20 | 130 | 45 | A1 (50%) |
| 10. | 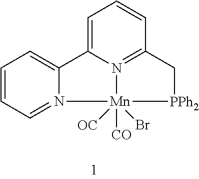 | 2/2 | 30 | 130 | 24 | A1 (51%) |
| 11. | 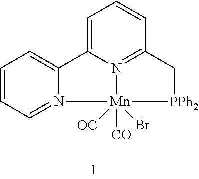 | 2/2 | 20 in dioxane | 130 | 40 | A1 (35%) |
| 12. | 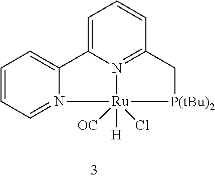 | 1/1 | 9.5 | 130 | 65 | A1 (70%) B (30%) |
| 13. | 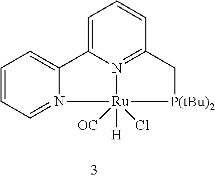 | 2/2 | 20 | 130 | 44 | B (99%) |
| 14. | 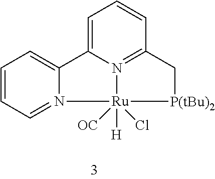 | 1/1 | 20 | 130 | 40 | B (99%) |
| 15. | | 0.1/1 | 65 | 145 | 68 | A1 (35%) |
| | | | | | | B (65%) |
| 16. | | 1/2 | 20 | 130 | 40 | B (99%) |
| 17. | | 1/2 | 10 | 130 | 40 | A1 (70%) |
| | | | | | | B (30%) |
| 18. | | 2/4 | 10 | 130 | 60 | B (99%) |

For entries 1-11, substrate (1 mmol), cat (as specified), base (as specified), THF (dioxane for entry 11) (4 mL), H2 pressure, temp, and time as specified.
For entries 12-18, 0.5 mmol of substrate was used, THF 4 mL.

### Example 2

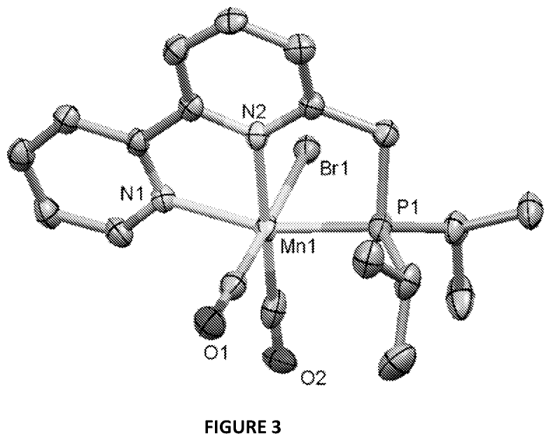

Synthesis and Characterization of Mn(CO)₂PNN^{iPr}Br (FIG. 3)

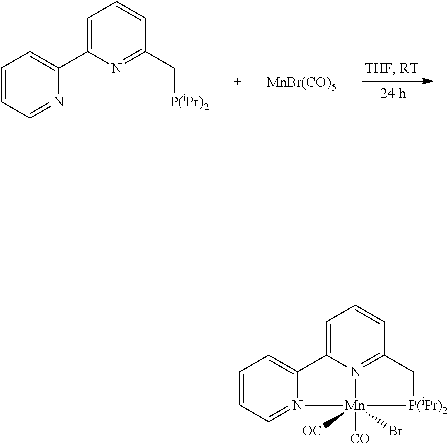

To a solution of the ^{iPr}PNN ligand (prepared according to a T. Zell et al Inorg. Chem. 2013, 52, 16, 9636-9649 (290 mg, 1.01 mmol) in 5 mL THF was added under nitrogen atmosphere (glove box) an orange solution of Mn(CO)₅Br (275 mg, 1 mmol) in 10 mL THF and the reaction mixture was kept stirring at room temperature for 24 h (Note: The CO gas liberated needs to be removed occasionally in vacuo). The solution was evaporated in vacuo. The solid residue was washed with pentane (10×3 mL), which on evaporation gave a dark brown solid product in 84% (400 mg) yield. The brown crude product was dissolved in THF (15 mL), the solution was filtered and concentrated, layered with pentane and kept in the refrigerator (−30° C.) to obtain dark brown crystals of the pure complex. (FIG. 3).

³¹P NMR (121 MHZ, Chloroform-d) δ 99.58.

¹H NMR (300 MHz, Chloroform-d) δ 9.48-9.25 (bs, 1H), 7.93 (s, 2H), 7.83 (s, 2H), 7.52 (d, J=5.3 Hz, 1H), 7.38 (s, 1H), 3.90-3.51 (m, 2H), 3.06 (dd, J=14.3, 6.8 Hz, 1H), 2.57-2.39 (m, 1H), 1.51 (dd, J=16.5, 7.1 Hz, 3H), 1.30 (dt, J=14.9, 8.5 Hz, 6H), 1.16 (dd, J=14.1, 6.9 Hz, 3H).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A process for the preparation of 1-(4-(benzyloxy)-3-(hydroxymethyl)phenyl)-2-(tert-butylamino)ethanol: 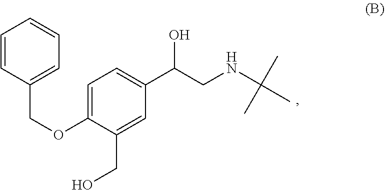 [SMILES: CC(C)(C)NCC(O)c1ccc(OCc2ccccc2)c(CO)c1]
wherein the process comprises reacting (E)-methyl 2-(benzyloxy)-5-(2-(tert-butylimino)acetyl)benzoate: 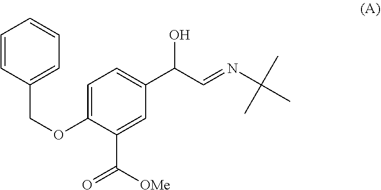 [SMILES: COC(=O)c1cc(C(O)/C=N/C(C)(C)C)ccc1OCc1ccccc1]
with a catalyst represented by the structure of formula Ia or Ia′ under hydrogen pressure: 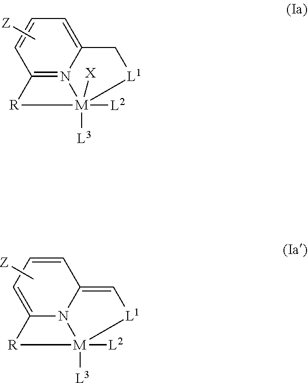
wherein,
M is a transition metal Ru(I) or Mn(II);
R is CH₂L⁴ wherein L⁴ is coordinated with the metal; or R is substituted or unsubstituted pyridyl group, wherein the nitrogen of the pyridyl group is coordinated with the metal;
L¹ is (PR^{a}R^{b}), (NR^{a}R^{b}), imine; oxazoline, sulfide (SR^{a}), heteroaryl containing at least one heteroatom selected from nitrogen and sulfur; or a N-heterocyclic carbene represented by the structures: 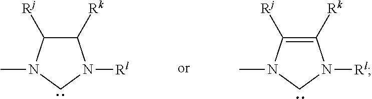
if M is Mn(I), L² and L³ are each independently a mono-dentate two-electron donor selected from the group consisting of CO, PR^{a}R^{b}R^{c}, P(OR^{a})(OR^{b})(OR^{c}), NO⁺, NR^{a}R^{b}R^{c}, AsR^{a}R^{b}R^{c}, SR^{a}R^{b}, nitrile (RCN), isonitrile (RNC), PF₃, CS, heteroaryl, tetrahydrothiophene, alkene or an alkyne;
if M is Ru(II), L² is a mono-dentate two-electron donor selected from the group consisting of CO, PR^{a}R^{b}R^{c}, P(OR^{a})(OR^{b})(OR^{c}), NO⁺, NR^{a}R^{b}R^{c}, AsR^{a}R^{b}R^{c}, SR^{a}R^{b}, nitrile (RCN), isonitrile (RNC), PF₃, CS, heteroaryl, tetrahydrothiophene, alkene, alkyne; and L³ is H, halide, OCOR^{X}, OCH₂Q, OCOCF₃, OSO₂R^{X}, OSO₂CF³, CN, OR^{X}, N(R^{X})₂ or R^{X}S; wherein Q is hydrogen, alkyl, cycloalkyl, aryl, heterocyclyl, heteroaryl, alkylcycloalkyl, alkylaryl, alkylheterocyclyl or alkylheteroaryl;
L⁴ is (PR^{a}R^{b}), (NR^{a}R^{b}), imine; oxazoline, sulfide (SR^{a}), heteroaryl containing at least one heteroatom selected from nitrogen and sulfur; (AsR^{a}R^{b}), or a N-heterocyclic carbene represented by the structures: 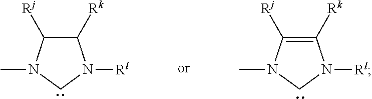
R^{j}, R^{k} and R^{l} are substituents of a N-heterocyclic carbene wherein each independently H, alkyl, cycloalkyl, aryl, heterocyclyl, heteroaryl, alkylcycloalkyl, alkylaryl, alkylheterocyclyl or alkylheteroaryl;
X is H, halide, OCOR^{X}, OCH₂Q, OCOCF₃, OSO₂R^{X}, OSO₂CF₃, CN, OR^{X}, N(R^{X})₂ or R^{X}S; wherein Q is hydrogen, alkyl, cycloalkyl, aryl, heterocyclyl, heteroaryl, alkylcycloalkyl, alkylaryl, alkylheterocyclyl or alkylheteroaryl;
R^{X}is hydrogen, alkyl, cycloalkyl, aryl, heterocyclyl, heteroaryl, alkylcycloalkyl, alkylaryl, alkylheterocyclyl or alkylheteroaryl;
Z represents zero, one, two or three substituents wherein each such substituent is independently selected from the group consisting of alkyl, cycloalkyl, aryl, heterocyclyl, heteroaryl, alkylcycloalkyl, alkylaryl, alkylheterocyclyl, alkylheteroaryl, halide, nitro, amide, ester, cyano, alkoxy, alkylamino, arylamino, an inorganic support and a polymeric moiety; or Z forms a fused aromatic or heterocyclic ring with the nitrogen based ring; and
R^{a}, R^{b} and R^{c} are each independently H, alkyl, cycloalkyl, aryl, heterocyclyl, heteroaryl, alkylcycloalkyl, alkylaryl, alkylheterocyclyl or alkylheteroaryl.

2. The process of claim 1, wherein the catalyst is represented by the structure of formula Ib or Ib′:

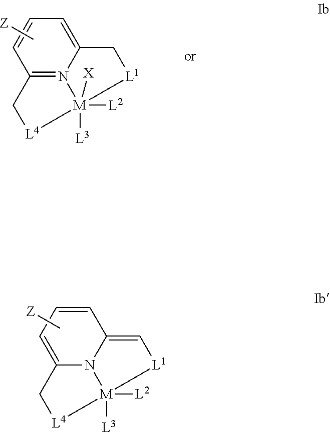

3. The process of claim 1, wherein the catalyst is represented by the structure of formula Ic or Ic′:

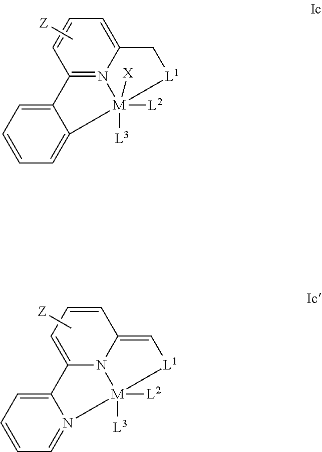

4. The process of claim 1, wherein the catalyst comprises Mn and is represented by the structure of formula Id, Id′, Ie or Ie′:

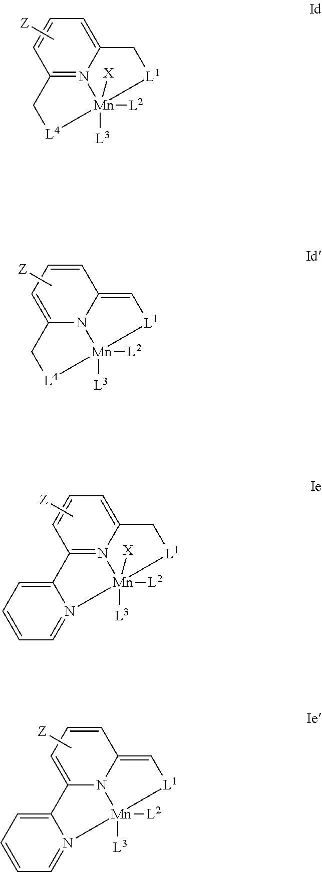

5. The process claim 1, wherein the catalyst comprises Ru and is represented by the structure of formula If, If′, Ig, Ig′:

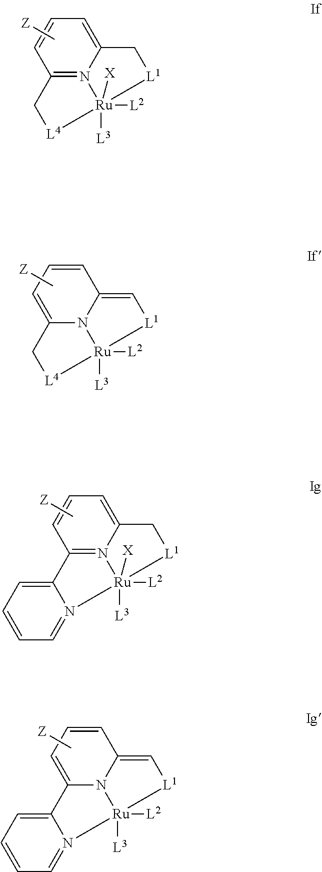

6. The process of claim 1, wherein the catalyst is selected from catalysts 1-5:

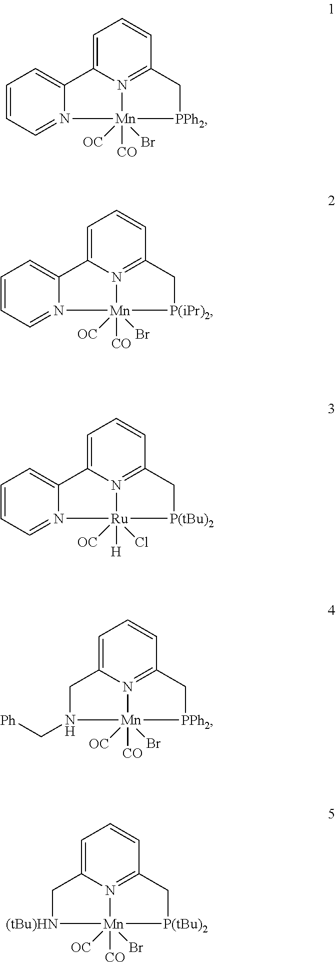

7. The process of claim 1, wherein the hydrogen pressure is between 10-70 bars.

8. The process of claim 7, wherein the hydrogen pressure is between 30-50 bars.

9. The process of claim 1, wherein the reaction is conducted in the presence of a strong base.

10. The process of claim 1, wherein the reaction is conducted with the exclusion of air.

11. The process of claim 1, wherein the reaction is conducted at a temperature between 120-150° C.

12. The process of claim 9, wherein the molar ratio between the catalyst and the base is 1:1.

13. The process of claim 1, wherein the molar ratio between (E)-methyl 2-(benzyloxy)-5-(2-(tert-butylimino)acetyl)benzoate (A) and the catalyst is between 100:1 to 20:1.

14. A process for the preparation of Salbutamol, wherein the process comprises reduction of Salbutamol intermediate 1-(4-(benzyloxy)-3-(hydroxymethyl)phenyl)-2-(tert-butylamino)ethanol: 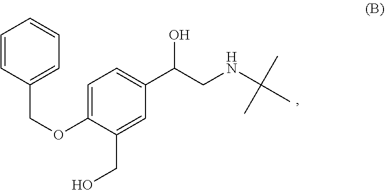 [SMILES: CC(C)(C)NCC(O)c1ccc(OCc2ccccc2)c(CO)c1]
wherein the Salbutamol intermediate is prepared according to the process of claim 1.

15. The process of claim 2, wherein the catalyst is represented by the structure of formula Id, Id′.

16. The process of claim 3, wherein the catalyst is represented by the structure of formula Ie or Ie′.

17. The process of claim 2, wherein the catalyst is represented by the structure of formula If, If′.

18. The process of claim 3, wherein the catalyst is represented by the structure of formula Ig, Ig′.
